(19) United States
(12) Patent Application Publication
(10) Pub. No.: US 2025/0291422 A1
(43) Pub. Date: Sep. 18, 2025

(54) SYSTEMS AND METHODS FOR DESIGN OF DEVICE TO INFER HAND INTERACTIONS BY FUSING DEPTH SENSING AND BIOACOUSTICS

(71) Applicants: Yatiraj Shetty, Tempe, AZ (US); Troy McDaniel, Chandler, AZ (US)

(72) Inventors: Yatiraj Shetty, Tempe, AZ (US); Troy McDaniel, Chandler, AZ (US)

(73) Assignee: Arizona Board of Regents on Behalf of Arizona State University, Tempe, AZ (US)

(21) Appl. No.: 19/079,344

(22) Filed: Mar. 13, 2025

Related U.S. Application Data
(60) Provisional application No. 63/564,640, filed on Mar. 13, 2024.

(51) Int. Cl. G06F 3/01; A61B 8/08; G01S 17/08

(52) U.S. Cl. CPC G06F 3/017; A61B 8/08; G01S 17/08

(57) ABSTRACT

Examples of a wrist-band device are disclosed that combines depth sensing and bioacoustic signals for precise hand interaction detection. The device incorporates a 2D optical depth sensor and an acoustic vibration sensor, facilitating accurate interpretation of hand movements in two modalities: hand shapes and bioacoustic signals. This multi-modal sensing approach allows the device to accurately detect and interpret hand movements.

### CROSS REFERENCE TO RELATED APPLICATIONS

This is a non-provisional application that claims benefit to U.S. Provisional Application Ser. No. 63/564,640, filed on Mar. 13, 2024, which is herein incorporated by reference in its entirety.

### GOVERNMENT SUPPORT

This invention was made with government support under 2142774 awarded by the National Science Foundation. The government has certain rights in the invention.

### FIELD

The present disclosure generally relates to wearable devices; and in particular to a device for gesture tracking and configured for accurately classifying discrete and continuous hand gestures and actions.

### BACKGROUND

The advent of wearable devices has revolutionized the way we interact with technology, particularly through hand interaction detection. Hand interactions, encompassing gestures and hand-object interactions, are integral to human-computer interaction (HCI), spanning a wide range of applications, from gesture recognition to context-awareness in diverse domains. Despite their significance, accurately capturing the nuanced expressivity of these interactions, especially microgestures, remains an enduring challenge for modern sensing systems. For instance, while external depth cameras have been extensively employed for hand tracking, they encounter issues of occlusion and may not always be feasible to deploy in various environments.

Wrist-worn camera approaches have been explored, yet they encounter various challenges, including occlusion, environmental lighting, complex algorithm design, and privacy concerns. As a result, researchers have turned to alternative line-of-sight sensors such as infrared time-of-flight and ultrasonic sensors. However, these solutions often necessitate the integration of multiple sensors to address occlusion, leading to intricate hardware designs, calibration requirements, and latency issues. To surmount these limitations, the research community has also delved into non-line-of-sight sensing methodologies, leveraging physiological signals to classify gestures and detect objects. These techniques encompass diverse approaches, such as inertial sensors, skin capacitance changes, electromyography (EMG), force myography, and skin deformation, among others. Despite their potential, these methods often involve complex signal conditioning, susceptibility to environmental or skin conditions, bandwidth limitations, and limited classification of intricate finger gestures. More recently, researchers have explored sensor fusion approaches that combine multiple modalities to enhance inference capabilities. While promising, these approaches still face challenges related to stability in noisy environments, complexity in algorithm design, and limitations in gesture classification sets.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1A is an illustration including a system diagram of a device for inferring hand interactions by infusing depth sensing and bioacoustics.

FIG. 1B is a photograph of an example implementation of the device described herein mounted on a wrist band.

FIG. 1C is a photograph of a top view of the example of the device from FIG. 1B

FIG. 2 is a sampling of point clouds generated by the device.

FIG. 3A is a point cloud during a first gesture performed when the field of view is not ‘compact.’

FIG. 3B is a point cloud during a first gesture performed when the field of view is ‘compact.’

FIG. 4 is a point cloud during a finger pinch gesture indicating the ellipticity of the shape through a wrist view.

FIG. 5 is an illustration demonstrating directional feedback associated with an example calibration algorithm that can be implemented to calibrate the device described herein.

FIG. 6A-6B are point cloud images that illustrate the different between a compact (5B) and non-compact (5C) image.

FIG. 7 illustrates various positions along the wrist.

FIG. 8 illustrates views of the depth image corresponding to the positions on the wrist indicated in FIG. 7.

FIG. 9 is an example method or process that can be implemented for inferring hand interactions by infusing depth sensing and bioacoustics according to functionality described herein.

Corresponding reference characters indicate corresponding elements among the view of the drawings. The headings used in the figures do not limit the scope of the claims.

### DETAILED DESCRIPTION

The present disclosure relates to examples of a device for novel gesture tracking device with the capability of accurately classifying discrete and continuous hand gestures and actions. The device can be mounted on a wristband, and can include a depth sensor such as a two-dimensional (2D) 8×8 time of flight (TOF) sensor and an acoustic vibration sensor, both controlled by a small microcontroller. Leveraging the depth sensor, the device generates a three-dimensional (3D) representation of the hand region of interest within a 45-degree field of view (FOV) from the acquired depth data. Even with low resolution (64-pixel depth image), the transformed data captures intricate details of hand deformations, enabling precise inferences through one or more machine learning models/algorithms. In addition to depth sensing, the device captures bioacoustic vibration signals propagating through the hand, detected by the acoustic vibration sensor. This multimodal approach enhances the device's capabilities, opening avenues for intuitive applications, such as gesture-based control of smart devices or cameras.

### INTRODUCTION

Examples of a device for gesture tracking described herein center on the potential of fusing depth sensing and bioacoustic signals to precisely infer hand movements and interactions. To achieve a more robust approach, examples focus on two critical outcomes: performing 3D hand shape analysis and discriminating bioacoustic signals based on their source of vibration. To realize this vision, a 2D depth sensor and an acoustic vibration sensor are leveraged, which were thoughtfully chosen to mitigate environmental effects, enhance signal-to-noise ratio (SNR), and adhere to the wrist-worn wearable form-factor requirements. This strategic selection significantly simplifies algorithm development and empowers accurate inferences.

More specifically, the inventive concept revolves around novel gesture tracking and can take the form of a device comprising two key sensors: a depth sensor such as a Time-of-Flight (TOF) 8×8 multizone ranging sensor (VL53L5CX) and one or more of an acoustic sensor (e.g., Sonion VPU14DB01). The device is powered by a high-performance microcontroller for data acquisition. The acquired data can be transmitted via USB C serial connection or Bluetooth low energy protocol.

A primary goal of this device is not comprehensive hand gesture tracking but rather the inference of hand states within a limited field of view of 45 degrees. To achieve this, machine learning can be leveraged on multimodal sensing data generated by the system. A low-resolution 2D image of the hand captured by the depth (TOF) sensor is utilized to generate point cloud data. This gives important information about the hand shape in 3D. The acoustic sensor plays a crucial role in capturing relevant audio signals. To reduce effects of background noise, acoustic sensors that are sealed or otherwise protected can be implemented to function like a contact sensor to improve the signal-to-noise ratio (SNR).

Wrist-worn sensors often encounter the sensor-shift issue, requiring frequent calibration when the device is removed and re-worn. To address this challenge, the inventive concept incorporates a calibration algorithm that analyzes the shapes generated by the point clouds and guides the user to maintain a consistent location of the sensor relative to the hand region of interest. This calibration process ensures reliable and precise tracking of hand actions.

The intended application of this device is as a new form of controller for gesture-based interfaces, ensuring an enhanced user experience while prioritizing user privacy. Privacy concerns are mitigated by the limited range and resolution of the depth sensor. Additionally, the technology is envisioned to complement previous inventions, such as SleeveSight (a smart haptic sleeve) and Peractiv (a smart wrist camera), thereby enhancing their sensing capabilities.

### Example Description

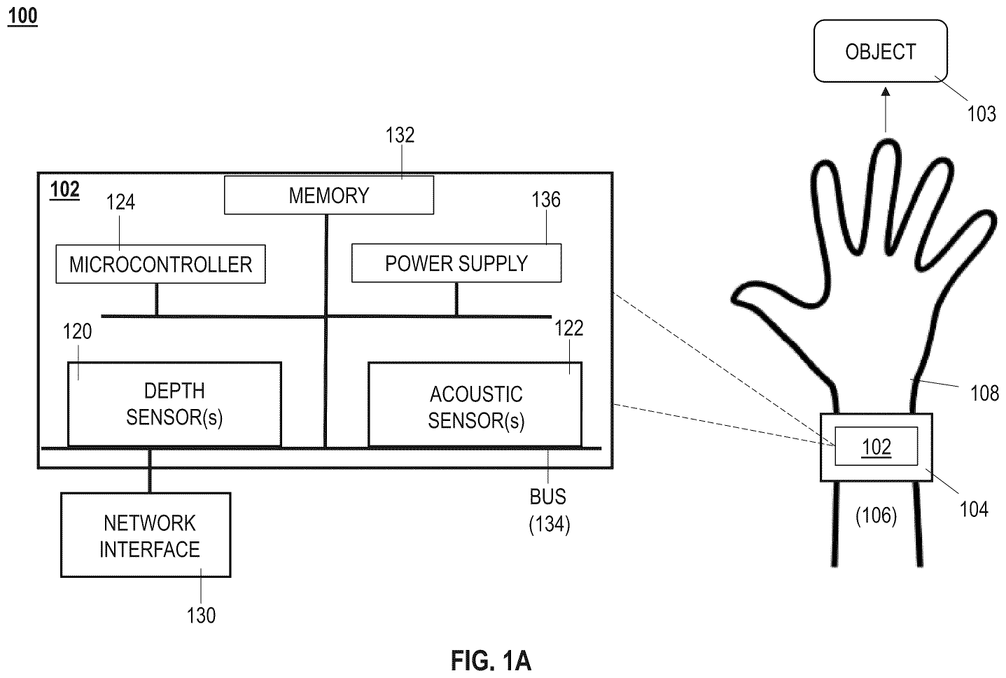

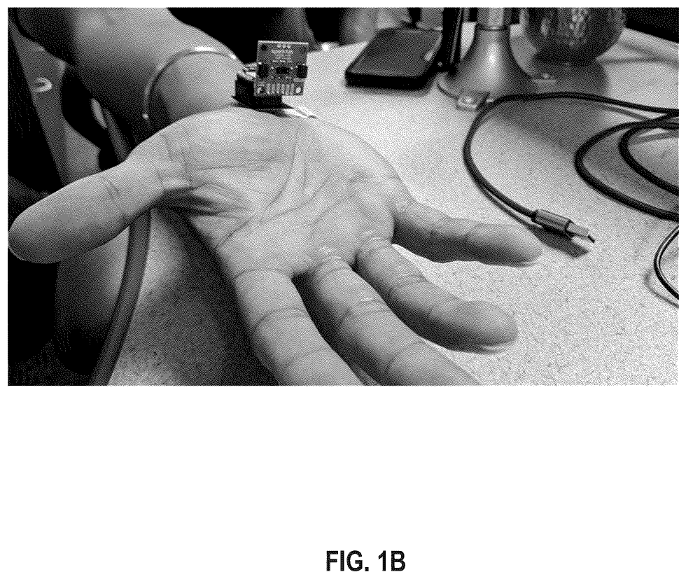

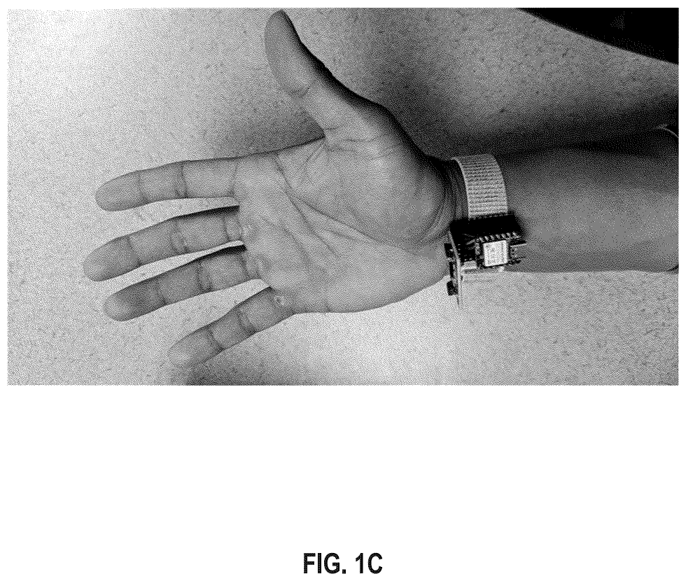

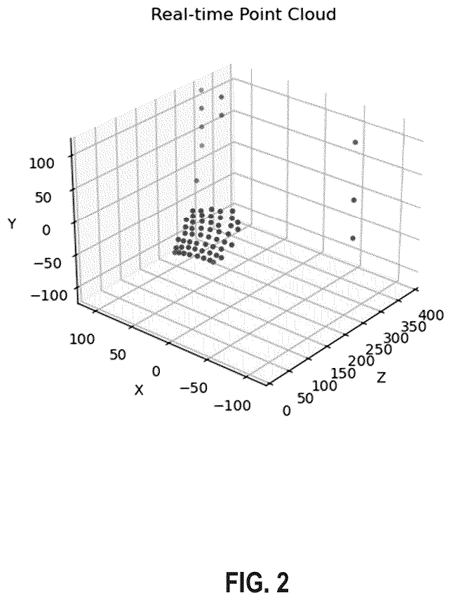

Referring to the system 100 of FIGS. 1A-1C, in one example, the inventive concept can take the form of a wearable device 102 for gesture tracking and/or inferring hand interactions relative to an object 103 or otherwise. The device 102 can be mounted to, or engageable to a wrist band 104 that a user can wear about a wrist 106 of a hand 108 (example prototypes shown in FIGS. 1B-1C). In this manner, the device 102 is compact and wrist-worn, making it portable and convenient for everyday use. The device 102 can be configured to capture and/or track discrete gestures and continuous hand actions, with one aim of providing intuitive gesture-based control for smart devices and cameras.

### Components of the Device:

(1) Depth Sensor 120 (e.g., Time-of-Flight (TOF) Sensor): A core sensing element of the device 102 is at least one depth sensor 120. In some examples, the depth sensor 120 can include a 2D 8×8 multizone ranging TOF sensor (VL53L5CX). The depth sensor 120 can be strategically placed on the ventral side of the wrist, within the wrist band 104. The depth sensor 120 captures low-resolution 2D depth images of the hand within a limited field of view of 45 degrees.

(2) Acoustic Sensor 122 (e.g., Sonion VPU14DB01): The device 102 also includes at least one acoustic sensor 122, which can function as an acoustic vibration sensor. The acoustic sensor 122 can be hermetically sealed to ensure robustness against background noise and can capture relevant audio signals that propagate through the hand during gestures and hand-object interactions. Because this sensor functions like a contact microphone the environmental noise travelling through air is not picked up by the device. This ensures that the signals acquired through acoustic sensor have high SNR.

(3) Microcontroller 124: Data acquisition and processing are managed by a high-performance microcontroller 124 with an in-built IMU (seeed studio xiao nrf52840 sense). The microcontroller 124 serves as the central processing unit, facilitating the conversion and fusion of data from the depth sensor 120 and acoustic sensor 122. In some examples IMU signals can further be fed to the microcontroller 124 to supplement the capabilities of the device 102.

The device 102 can be configured or based on principles of multimodal sensing and combines depth information (from TOF sensor) and bioacoustic information (from the acoustic sensor) to generate accurate inferences: (1) dynamic hand gestures (2) microgestures in mid-air or when grasping objects (3) force/squeeze of fingers or palm (3) object detection.

As further shown, the device 102 can also include one or more network interfaces 130 (e.g., wired, wireless, PLC, etc.), a memory 132 interconnected by a system bus 134, as well as a power supply 136 (e.g., battery, plug-in, etc.). Network interface(s) 130 include the mechanical, electrical, and signaling circuitry for communicating data over the communication links coupled to a communication network. Network interfaces 130 are configured to transmit and/or receive data using a variety of different communication protocols. As illustrated, the box representing network interfaces 130 is shown for simplicity, and it is appreciated that such interfaces may represent different types of network connections such as wireless and wired (physical) connections. Network interfaces 130 are shown separately from power supply 136, however it is appreciated that the interfaces that support PLC protocols may communicate through power supply 136 and/or may be an integral component coupled to the power supply 136.

Memory 132 includes one or more storage locations that are addressable by the microcontroller 124 and network interfaces 130 for storing software programs and data structures associated with the embodiments described herein. In some embodiments, device 102 may have limited memory or no memory (e.g., no memory for storage other than for programs/processes operating on the device and associated caches). Memory 132 can include instructions executable by the microcontroller 124 that, when executed by the microcontroller 124, cause the microcontroller 124 to implement aspects of the system 100, functionality, and other aspects outlined herein. Microcontroller 124 comprises hardware elements or logic adapted to execute the software programs (e.g., instructions) and manipulate data structures. The device 102 can include an operating system, portions of which are typically resident in memory 132 and executed by the microcontroller 124, functionally organizes device 102 by, inter alia, invoking operations in support of software processes and/or services executing on the device 102.

### Device Fabrication and Assembly:

The device 102 can be assembled into the wrist band 104, allowing for easy and comfortable wearability on the wrist 106. In some examples, the depth sensor 120 and acoustic sensor 122 are carefully integrated into the wrist band 104 to ensure optimal sensor placement for accurate data capture. The depth sensor 120 can be set in a position in the volar region of the wrist 106 to ensure good view of the hand 108. The acoustic sensor 122 can be directly attached to the skin to pick up bioacoustic signals emanating from hand interactions (gesture and hand-object interactions). The microcontroller 124 can be securely housed within the wrist band 104, along with necessary circuitry for data transmission.

### Sensor Fusion and Data Processing:

Upon activation, the depth sensor 120 captures depth data, converting it into a point cloud representing the 3D shape of a region of interest relative to the hand 108. This point cloud enables the creation of a 3D representation of the position of the hand 108 and deformation. Positioned on the volar side of the wrist 106, the system 100 gains a comprehensive view of thumb-to-finger interactions, facilitating accurate inferences on dynamic hand gestures after appropriate training.

Concurrently, the acoustic sensor 122 captures bioacoustic vibration signals generated during interactions of the hand 108 that are traveling through the skin.

The utilization of both depth (e.g., TOF) and bioacoustic signals allows for hand gesture detection even when the depth sensor 120 is occluded by an object. Remarkably, during research, it was observed that the device 102 could capture microgestures performed during mid-air gestures and grasped object scenarios. The microcontroller 124 receives the data streams from both the depth sensor 120 and acoustic sensor 122 and can process them using various machine learning algorithms for inference. This multimodal sensor fusion enhances the accuracy and robustness of hand interaction detection, enabling the device 102 to precisely infer minute hand gestures and actions.

### Calibration Algorithm:

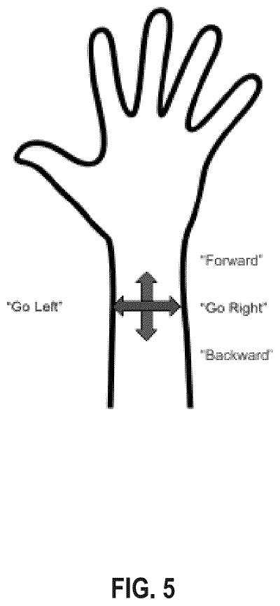

Referring to FIGS. 5-8, an example calibration algorithm is described for identifying an optimal placement location and/or position for a depth sensor 120 along the wrist.

Problem: It is a well-known problem with wrist worn sensors that they need frequent calibration to maintain good inference. This problem happens because the location of the sensor may shift when re-worn after initial use. Within the context of the current sensing system (100) this problem could be broken down into the following questions:
1. How to find the best view for the placement of the depth sensor 120 along the wrist?
2. How to maintain the consistency of location during subsequent wearing?

Overview: One solution is to automatically give users the feedback to move the depth sensor 120 to an appropriate location on the wrist 106 when mounting. It should be simple directional feedback (FIG. 5) to make it easier for the user to find the correct position.

The user requirements for the algorithm can be summarized as follows.
(1) Initial run: direct the user to a ‘good position’ and record the final position. The program guides the user forward/backward and left and right.
(2) Subsequent runs: direct the user to move the device to the previous position.

### Algorithm Design

### Defining the ‘Good View’

As the depth sensor 120 being used can be a line of sight depth sensor, it is prone to occlusion or the region of interest being out of view. Hence a good view entails a position where the region of interest (i.e., palm and finger) is maximally viewable even when various kinds of gestures are performed. Based on heuristic evaluation of the sensor placement the following functional requirements could be ascertained.

In order to have a ‘good position’, the depth sensor 120 should be placed to accommodate the following.
1. The field of view captures the maximum region of hand
2. The image is compact; i.e., there are no wasted pixels.
3. Records the location of the image so that on re-wear the algorithm could direct it back to the same position.

And to achieve this the following design constraints should be considered:
As the intention is to view mid-air hand gestures, the depth values from the depth sensor 120 could be clipped to a certain range.
Only the volar region of the forearm close to the wrist needs to be considered as design space.

### Image Exploration

### Using the ‘Fist’ Gesture to Calibrate the Device.

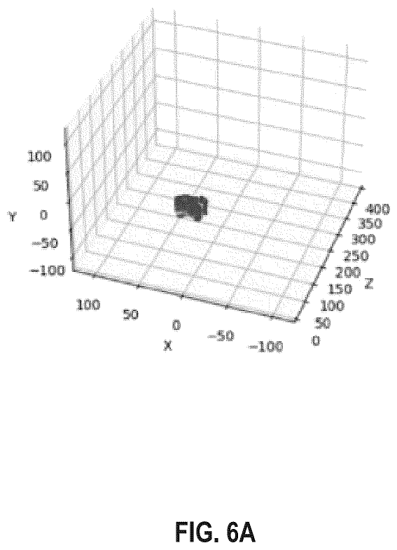

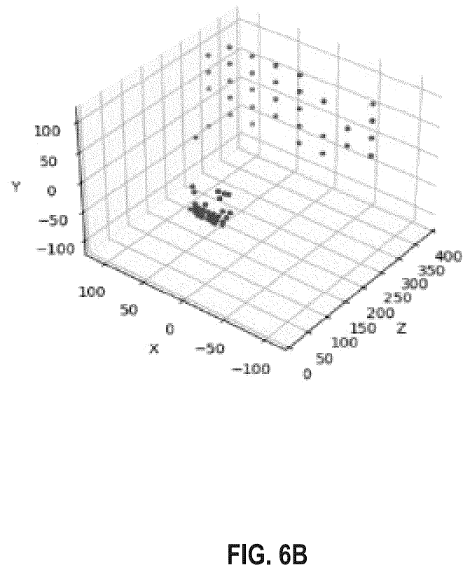

The compactness of shape refers to how close the points are to each other. In the case of the view of first gesture, the more compact an image is, the better it is able to capture the variations when the hand is deformed (i.e., when it performs a gesture). FIGS. 6A-6B show the difference between a compact (6A) and non-compact (6B) image. The images are shown in point cloud format for better visualization.

In simple terms, the more compact an image the better the resolution of gestures is captured by the depth sensor 120.

View of the First Gesture from Different Locations Along the Wrist

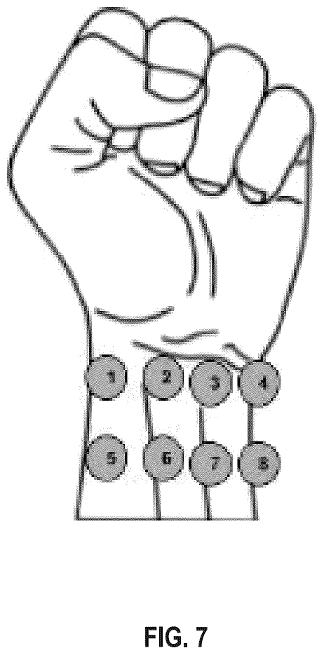

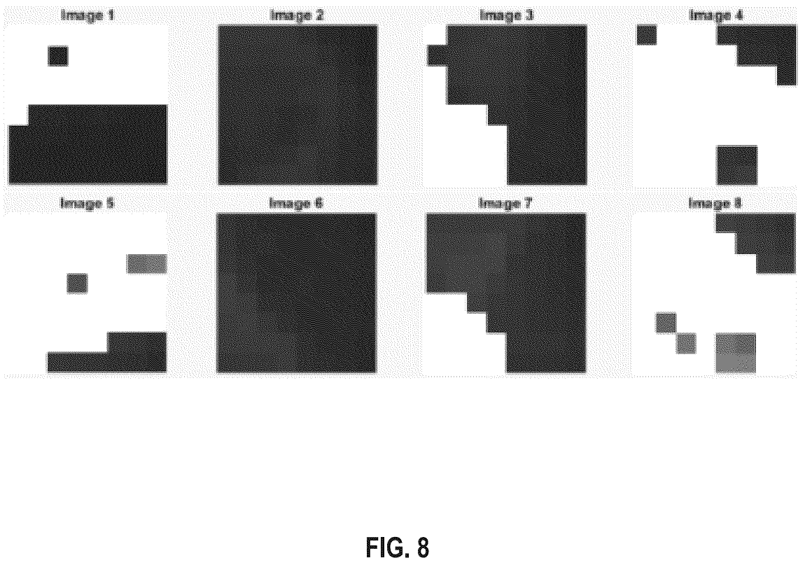

FIG. 7 shows possible placement locations of the depth sensor 120 along the wrist 106, and FIG. 8 illustrates depth images corresponding to the placement locations in FIG. 7. It should be noted that the images in FIG. 7 are presented in grayscale without filtering. Though image 2 and 6 of FIG. 7 appear similar the average depth values of pixel in image 2 are smaller than in the image 6 as it is closer to the fist.

### DISCUSSION

Based on the above observations, the problem of finding the optimal location could be reduced to a simple blob detection and tracking problem. After basic image filtering steps, blob region properties like region size and convexity could be used to track the location of blobs with respect to the image frame.

The calibration algorithm can include the following steps for initial run:
1. Place the depth sensor (120) in a random position on the wrist.
2. Grab depth image
3. Basic image filtering: convert to grayscale, spatial filtering and thresholding to identify the first region.
4. Blob detection and calculation of region properties
5. If the blob is o-center, direct the user to move left or right.
6. If the blob is far away (i.e. image is not compact), direct the user to move the sensor forward.
7. Repeat steps 1-6 until the optimal position is found. Then record the blob values for future reference.

For subsequent runs, the reference values can be used to direct the depth sensor 120 to a consistent position. Advantages of this approach include implementation of a simple algorithmic and user feedback design, as well as consistent location positioning for the device 102.

The calibration algorithm addresses two issues:
1. Maximum utilization of the field of view of TOF sensor.
2. The sensor-shift issue is commonly encountered in wrist-worn sensors.

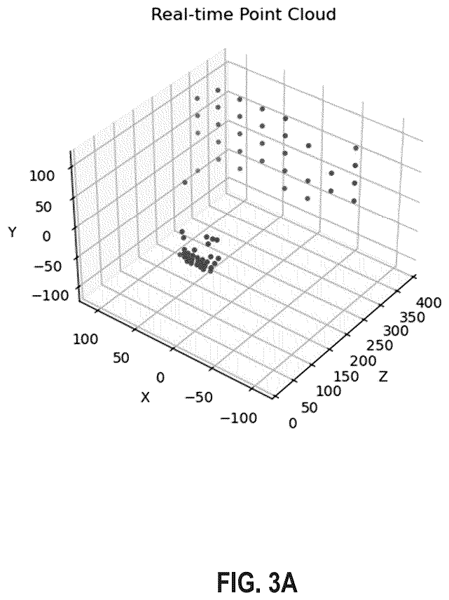

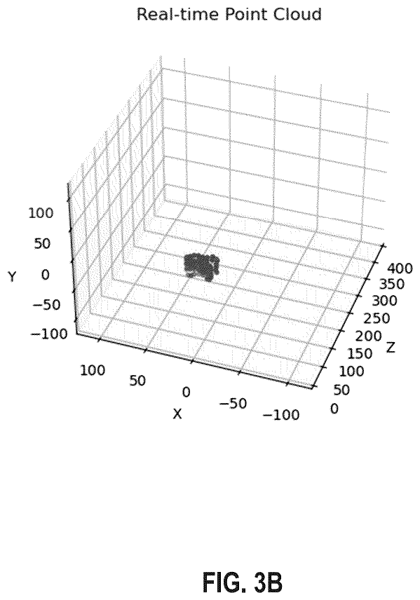

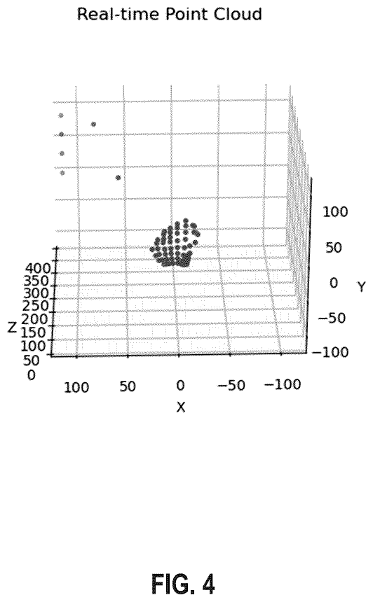

One important thing that was noticed during pilot studies is that during a specific hand gesture (for example first gesture, chosen because it is the smallest shape that utilized entire hand), the coverage of the point cloud is not complete i.e., less ‘compact’ (FIG. 3A) if the sensor is not positioned optimally. Theoretically to maximize resolution withing a limited FOV it is important to discriminate the maximum variance is shape possible. Hence if we can guide the positioning of the sensor such that the point cloud is more compact (shown in FIG. 3B) we would be able to track more minute details when continuous hand gestures are performed. Similarly it can be noted that the inclination of the shape is different for different point cloud shape, this is shown in FIG. 4.

The algorithm analyzes the shapes (convex hull) generated by the point clouds and provides feedback to the user to ensure a consistent location of the sensor relative to the hand region of interest based on shape descriptors (compactness, hull centroid distance and ellipticity) generated. Initially the user is asked to perform a simple gesture like a first or pinched fingers. The shape descriptors generated for this is unique and change predictably when the device is moved along the hand (longitudinal) or across the hand (lateral). Hence, they can be used to place the sensor consistently in the correct location. Calibration enhances the device's stability and accuracy, making it less susceptible to position variations caused by removal and re-wearing of the device.

### Advantages

Hand gesture recognition is an extensively researched field where various sensing modalities have been proposed to infer hand gestures, actions, and object interactions. Though various innovative sensing strategies have been proposed before (like using IMUs, mechanical sensors, electrical techniques), it is important to note that camera-based techniques are still by far the most accurate. But they suffer from three main issues: High processing requirements, occlusion, and privacy concerns. The inventive concept, e.g., device 102, intends to balance all these concerns by providing a solution that can use a single low resolution depth sensor (120) that has significantly low processing requirements as compared with a camera and does not record any intelligible objects or background information (which mitigated privacy concerns). Though there is still the problem of occlusion, it does not affect the efficacy of the device 102 for intended use cases: gesture recognition, tracking and hand action inferences.

While use of low resolution depth sensors have been demonstrated before, prior researchers used multiple sensing components, e.g., 16 depth sensors to generate very accurate hand and arm tracking. By comparison, the device 102 can be implemented with a single depth sensor 120 which is just good enough to perform the tasks intended for its use. This means the device 102 can operate at a higher update rate while being sufficiently accurate. Other researchers used two IR sensor and microphone to cleverly infer midair gestures. The two optical sensors only track general direction (not 3D shape of hand) of thumb movements and their system primarily uses a modified microphone to track continuous figure actions. The modified microphone used in the mentioned work has limited bandwidth.

In comparison the device 102 can be implemented using a single optical sensor and a single acoustic vibration sensor to perform similar inferences (and more complex inferences) and use only standard light weight algorithms. This, in future, could be easily incorporated into smartwatches or textile-based form-factors. This allows the on-board processor (e.g., microcontroller 124) to focus on other actions, for example controlling devices and even trigger cameras as needed. Note that these wide-ranging inferences (gestures, force sensing, etc.) have been performed in prior literature but using a different sensing principle that are usually susceptible to skin conditions.

### Testing & Evaluation

To evaluate the effectiveness of the proposed sensing approach, two crucial user studies were conducted focusing on the domain of microgestures. In the first study, expressive mid-air microgestures were explored, carefully selecting the design space based on previous work to present a challenging test case for the device 102. This study showcased the capabilities of the device 102 in capturing fine-grained finger interactions in both real-world and virtual environments. In the second study, participants engaged in hand-object microgestures, interacting with everyday objects while performing distinct microgestures. This evaluation sought to achieve two objectives simultaneously: identifying the held objects and classifying the microgestures performed on them. The combined results of these user studies provide comprehensive insights into the adaptability and feasibility of the inventive concept described herein in capturing diverse and expressive hand interactions.

The research associated with the inventive concept stands out from prior work in multi-modal sensing for hand gesture recognition through thoughtful sensor selection and the use of bioacoustic signals in challenging interactive scenarios, as demonstrated by the user studies. By harnessing the unique nature of bioacoustic signals propagating through our hands, more resilient inferences that are less susceptible to environmental noise and complementary to line-of-sight depth sensing are achieved. While it is acknowledged that this approach may not address all hand interaction challenges universally, its demonstrated accuracy in detecting complex microgestures showcases its potential to enhance existing hand interaction capabilities.

In general, it was demonstrated that the inventive concept provides the following key contributions:
1. Wrist-based multimodal sensing system that accurately infers complex hand interactions by combining two modes: hand shape analysis through depth-sensing and bioacoustic sensing.
2. A calibration algorithm designed to mitigate sensor-shift error inherent in wrist-worn sensing, thereby enhancing the accuracy of the system.
3. Comprehensive evaluation of the system's performance in challenging scenarios, including intricate mid-air and held-object microgestures, showcasing its versatility and robustness.
4. Demonstration of the wide-ranging sensing applications of this system beyond microgestures, underscoring its potential for expanding interaction paradigms in various contexts.

Microgestures, characterized by their detailed expressiveness, are frequently misinterpreted or overlooked by contemporary sensing systems. Given their complexity, microgestures were proposed as an optimal benchmark to assess the device's capabilities. The subject evaluation spans two essential hand interaction facets: mid-air gestures and interactions with held-objects. As results unfold it is hoped to find the device's adeptness not only in detecting microgestures but also in discerning a broader spectrum of hand interactions. These outcomes hint at the adaptability of the subject sensing approach, opening doors for expanded interaction paradigms in future wearable device research.

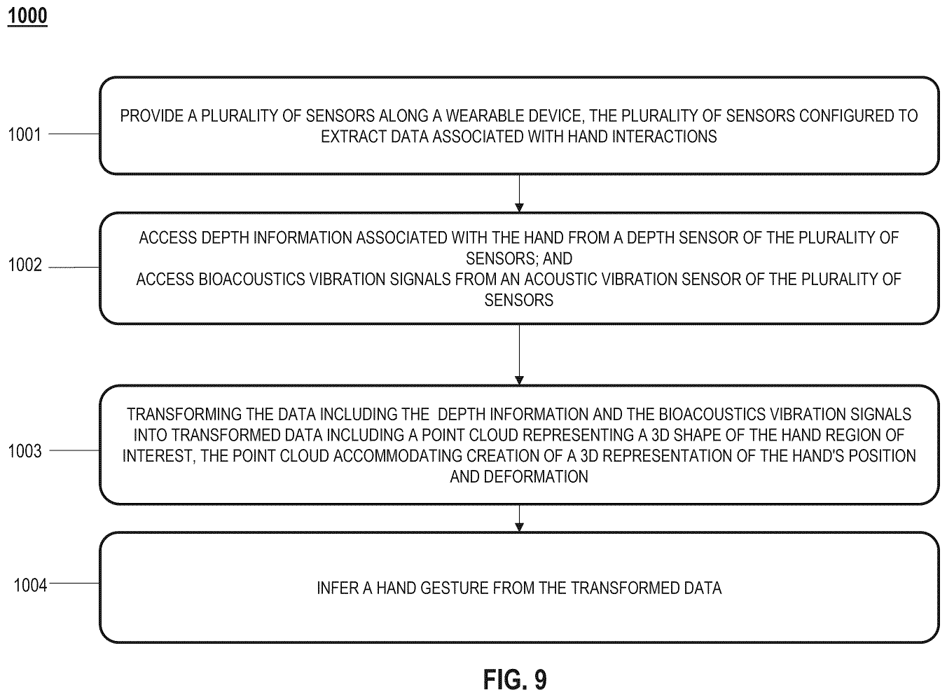

FIG. 9 illustrates an example process 1000 associated with the system 100 and device 102 described herein that can be implemented by any number or type of processing elements. As indicated in blocks 1001-1004, data from a plurality of sensors including depth information and bioacoustic vibrational signals can be accessed by a processor. Machine learning can be leveraged on multimodal sensing data generated by the system.

The processor transforms the data to a structure suitable for hand gesture inference. In particular, the processor accesses depth data from the depth sensor 120, converting it into a point cloud representing the 3D shape of a region of interest relative to the hand 108. This point cloud enables the creation of a 3D representation of the position of the hand 108 and deformation. Positioned on the volar side of the wrist 106, the system 100 gains a comprehensive view of thumb-to-finger interactions, facilitating accurate inferences on dynamic hand gestures after appropriate training.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system for gesture recognition, comprising:
a wearable device, comprising:
a plurality of sensors, including:
a depth sensor configured for placement along a ventral side of a wrist of a hand that captures images of the hand defining depth information within a predefined view, and
one or more acoustic sensors that generate bioacoustics information from audio signals that propagate through the hand during gestures and hand-object interactions; and
a microcontroller in operable communication with the plurality of sensors that facilitates conversion and fusion of the depth information from the depth sensor and the bioacoustics information from the one or more acoustic sensors to capture hand interactions including at least one gesture.

2. The system of claim 1, wherein the acoustic sensor captures the depth information, converting it into a point cloud representing a 3D shape of the hand region of interest, the point cloud accommodating creation of a 3D representation of the hand's position and deformation, and wherein concurrently the one or more acoustic sensors captures bioacoustic vibration signals generated during hand interactions.

3. The system of claim 1, further comprising:
a wristband, wherein the plurality of sensors and microcontroller are integrated into the wristband.

4. The system of claim 1, wherein for calibration of one or more sensors of the plurality of sensors the microcontroller finds an optimal location, such that the microcontroller:
accesses a position of the wearable device,
grabs a depth image,
conducts image filtering to identify a first region,
conducts blob detection and calculation of region properties, and iteratively repeat previous steps until an optimal position is found.

5. The system of claim 4, wherein if the blob is far away, the microcontroller issues an instruction to direct the user to move one or more of the plurality of sensors forward, and wherein if the blob is off-center the microcontroller issues an instruction to direct the user to move left or right.
